(19) United States
(12) Patent Application Publication
(10) Pub. No.: US 2025/0302944 A1
(43) Pub. Date: Oct. 2, 2025

(54) COMBINATION OF EPITOPES AND USE THEREOF, VACCINE CONSTRUCT, METHOD OF INDUCING AN IMMUNE RESPONSE, METHOD FOR THE IDENTIFICATION OF EPITOPES

(71) Applicant: FUNDAÇÃO ZERBINI, São Paulo (BR)

(72) Inventors: Jorge Elias KALIL FILHO, São Paulo (BR); Edecio Cunha NETO, São Paulo (BR); Daniela Santoro ROSA, São Paulo (BR); Keity Souza SANTOS, São Paulo (BR); Silvia Beatriz BOSCARDIN, São Paulo (BR); Marco Antonio STEPHANO, São Paulo (BR)

(21) Appl. No.: 18/850,231

(22) PCT Filed: Mar. 24, 2023
(86) PCT No.: PCT/BR2023/050101

§ 371 Date: Sep. 24, 2024

(30) Foreign Application Priority Data
Mar. 25, 2022 (WO) PCT/BR2022/050108

(51) Int. Cl. A61K 39/215; A61K 39/00; A61P 31/14; C07K 14/005; C12N 7/00; G16B 15/30

(52) U.S. Cl. CPC A61K 39/215; A61P 31/14; C07K 14/005; C12N 7/00; G16B 15/30; A61K 2039/572; A61K 2039/575; C12N 2770/20022; C12N 2770/20034

(57) ABSTRACT

COMBINATION OF EPITOPES AND USE THEREOF, VACCINE CONSTRUCT, METHOD OF INDUCING AN IMMUNE RESPONSE, METHOD FOR THE IDENTIFICATION OF EPITOPES The present invention refers to a combination of epitopes comprising at least eight T cell epitopes from the SARS-CoV-2, as well as the use of said combination (“set of epitopes”). Said epitopes are widely recognized by CD4+ T-lymphocytes of the overwhelming majority of COVID-19 convalescent individuals.

### REFERENCE TO SEQUENCE LISTING

A Sequence Listing conforming to the rules of WIPO Standard ST.26 is hereby incorporated by reference. Said Sequence Listing has been filed as an electronic document via PatentCenter in ASCII format encoded XML. The electronic document, created on Sep. 12, 2024, is entitled “P4898-seql-000001.xml”, and is 85, 696 bytes in size.

### FIELD OF THE INVENTION

The present invention refers to new epitopes used in combination which are recognized by CD4+ T-lymphocytes. In addition, the present invention refers to the uses of such epitopes and their combinations, particularly for the treatment or prevention of disorders caused by the SARS-CoV-2 virus.

Furthermore, the present invention refers to a method for the identification of epitopes used in a combination and methods for preventing an infection caused by the SARS-CoV-2.

Particularly, the present invention refers to a combination of epitopes comprising at least eight T cell epitopes from the SARS-CoV-2 in a construction with SARS-CoV-2 Spike protein receptor binding domain (RBD) monomer or dimer.

In addition, the present invention can be used to induce enhanced T cell responses to COVID-19 vaccine antigens and breakthrough infections. Thus, the present invention also describes the use of said combination for producing vaccines.

### BACKGROUND OF THE INVENTION

Although vaccines against the Covid-19 are already being used as an important response to the Covid-19 pandemic, it is well known by the society all around the world that the vaccines currently in use are not sufficient to eradicate the virus or even to prevent reinfection or transmission.

Moreover, since the begin of the vaccination in the end of 2021, the world is facing further challenges regarding the Covid-19 pandemic. It is still true that manufacturing, purchasing, and distributing to attain worldwide coverage are still formidable logistic and financial tasks, especially in low- to middle-income countries and remote areas. Therefore, an unequal distribution of vaccine between countries causes a poor vaccination coverage, far from the necessary percentage of immunized people to control the pandemic which also facilitates the occurrence of new variants.

Thus, there still an urgent need for vaccines which can induce a better immune response against the SARS-CoV-2 virus, which are reliable, low cost, easy to handle and store, but most importantly, are highly antigenic/immunogenic, including a better cellular (T-cell-mediated) immune response, which is more long-lasting than antibodies in coronavirus infections.

Documents already disclosed in the state of the art tried to provide a technical solution in terms of immune response to control the Covid-19 pandemic.

Document WO2021195286 discloses compositions and methods for treating and preventing coronaviruses, using a RBD polypeptide.

Document IN202011018845 discloses a codon-optimized nucleotide sequences designed to express the RBD of the Spike protein of SARS-CoV-2. The technical solution described in this document intends to identify small molecules or peptides with antiviral potential, for the development of an antigen-antibody-based diagnostic test, and for the discovery and validation of antibodies targeting SARS-CoV-2 RBD.

None of those documents were able to provide said technical solution.

Therefore, it is a goal of the present invention to provide a vaccine construct which induces a better cellular immune response.

In order to achieve said purpose, it is necessary to identify a set of SARS-CoV-2 T cell epitopes with wide population coverage which in a combination with a monomeric or dimeric RBD of the Spike protein from SARS-CoV-2 viruses is able to induce a higher cellular immune response in all of the population exposed to the virus.

The identification of such immunodominant peptides is not obvious. Moreover, given the nature of T cell epitope recognition, it is unlikely that a single T cell epitope may be recognized by all individuals. It is thus necessary to use a combination of immunodominant T cell epitopes to maximize coverage in a vaccine.

According to the in silico analysis, an increasing number of epitopes would allow recognition of a higher number of peptides per HLA-DRB1 and per individual, reaching a minimum of 10 peptides/HLA-DRB1 when using the complete set of peptides from SEQ ID NO: 1 to 19. An increasing number of recognized epitopes/individual increases the amplitude and coverage of the vaccination. The more peptides each vaccinee recognizes, the more difficult it is that mutations could jeopardize vaccine-induced protection to new mutant viruses. Complete coverage with ample breadth (number of T cell epitopes recognized per individual) is an expected emergent property of a combination of promiscuous HLA-binding epitopes. Each epitope individually will never be as antigenic/immunogenic in a genetically heterogeneous population as a combination of promiscuous, multiple HLA-DR-binding individual epitopes.

Thus, the present invention discloses a technical solution which is the identification and use of a set of SARS-CoV-2 peptides capable of binding multiple HLA class II molecules in a vaccine construct against the SARS-CoV-2 viruses.

Consequently, each individual would carry HLAS capable of presenting multiple such SARS-CoV2 peptides to T cells. This would allow that that the overwhelming majority of the vaccinated population will present T cell responses to multiple epitopes.

Moreover, in vaccines, CD8+ T cell epitopes are essential for the destruction of virus-infected cells and are instrumental for the control of subsequent infection or reinfection. The way to identify CD8+ T cell epitopes with wide population coverage is to search for peptides stably binding to HLA class I molecules/alleles covering the overwhelming majority of the population.

According to the method of identification of the present invention, it is possible to identify a number of peptides whose recognition is equivalent to the hundreds of peptides as taught in the state of the art but with much less resources as the present invention adopts a small number of peptides instead of hundreds.

### BRIEF DESCRIPTION OF THE SEQUENCE LISTING

The present invention will be more clearly understood upon reading the following non-restrictive detailed description and the Sequence Listing as presented herein.

### SUMMARY OF THE INVENTION

The present invention refers to a combination of epitopes comprising at least eight T cell epitopes from the SARS-CoV-2, as well as the use of said combination (“set of epitopes”). Said epitopes are widely recognized by CD4+ T-lymphocytes of the overwhelming majority of COVID-19 convalescent individuals.

Another object of the present invention is a vaccine construct comprising amino acid sequence of a single polypeptide.

Moreover, the present invention also embodies a method of inducing an immune response comprising administering the said vaccine construct.

Moreover, the present invention refers to a method for the identification of the best combination of SARS-CoV-2 T cell epitopes for a vaccine, allowing high coverage of the world population, which comprises the steps of:
a) selecting of the peptides from the entire SARS-CoV-2 proteome sequence, wherein said peptides bind to at least 50% of the HLA-DR molecules among in the bioinformatic server https://webs.iiitd.edu.in/raghava/propred/;
b) expanding of the sequences of said peptides selected at the N-terminal and C-terminal ends;
c) performing world population coverage studies based on frequencies of HLA molecules predicted to bind to peptides at the iedb.org website;
d) synthesizing of corresponding peptides;
e) confirming and testing for SARS-CoV-2-specific responses in sensitized individuals that have had mild COVID-19 disease (no hospitalization) and have recovered well.

Further object of the present invention is the use of the set of epitopes/combination of epitopes for producing vaccines constructs using a dimeric or monomeric RBD of the Spike protein of SARS-CoV-2 virus.

### BRIEF DESCRIPTION OF THE DRAWINGS

FIG. 1: FIG. 1 is a graph representing the world population coverage of all 19 peptides altogether, based on the frequencies of HLA molecules predicted to bind to the peptides as assessed by the IEDB population coverage at iedb.org. Results of the in silico analysis indicate that 99.6% of the world population could recognize at least one of the epitopes. Average hit means that on average each individual should recognize 29 HLA/epitope combinations. Pc90 means that 90% of the world population should recognize at least 15 HLA/epitope combinations.

FIG. 2: FIG. 2 shows cytokine release-based whole blood cytokine secretion high-throughput T cell assay with the 19 SARS-CoV-2 CD4+ T cell epitopes of the present invention. It shows that 42/45 (94%) of SARS-CoV-2 seropositive subjects display peptide-induced cytokine secretion indicative of T cell recognition of T cell epitopes, and none of the 16 seronegative subjects displayed T cell responses. This shows the specificity of the peptides for COVID-19-convalescent individuals.

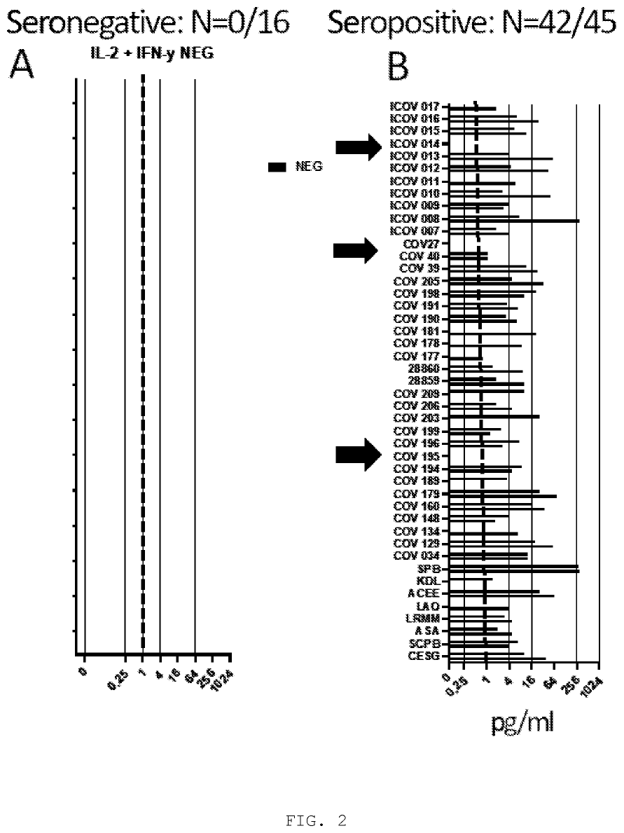

FIG. 3: FIG. 3 shows the ROC curve-production of IL-2 and IFN-γ in whole blood in response to the SARS-CoV-2 epitopes of the present invention.

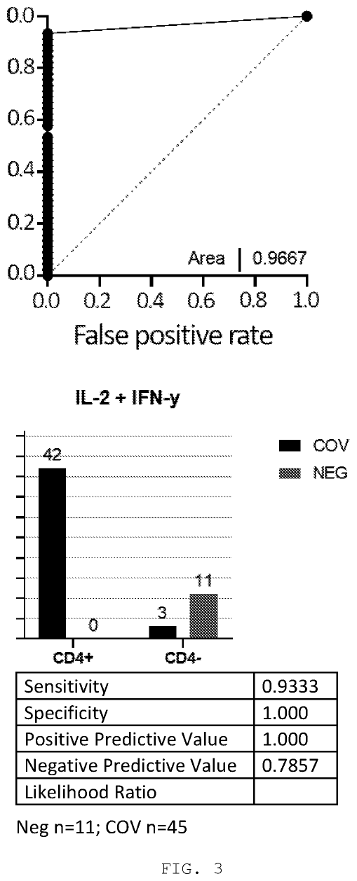

FIG. 4A/B: FIG. 4 shows the dimeric RBD protein derived from the Wuhan strain. FIG. 5A) Schematic drawing of the dimeric RBD protein. FIG. 5B) Map of the pcDNA3.1 plasmid containing the 2 tandem sequences of the RBD.

FIG. 5A/B: FIG. 5 shows the purification of dimeric RBD protein by nickel resin affinity chromatography. FIG. 5A) Purification chromatogram with IMAC-Ni resin. FIG. 5B) 7.5% SDS-PAGE gel under non-reducing conditions containing different fractions: 1. molecular weight marker; 2. pre-column dialyzed supernatant; 3. eluate after loading onto the column; 4. eluate after washing step; 5. eluate from elution fraction 1; 6. eluate from elution fraction 2; 7. eluate after column regeneration.

FIG. 6A/B: FIG. 6 shows the purification of dimeric RBD protein by ion exchange. FIG. 6A) Ion exchange purification chromatogram on S-Sepharose with saline concentration gradient up to 500 mM. FIG. 6B) 7.5% SDS-PAGE gel under non-reducing conditions containing different fractions: 1. pre-column dialyzed supernatant; 2. eluate after loading onto the column; 3. eluate after washing step; 4. molecular weight marker; 5. eluate from elution fraction 1; 6. eluate from elution fraction 2; 7. eluate from elution fraction 3; 8. eluate from elution fraction 4; 9. eluate from elution fraction 5; 10. eluate from the elution fraction 6.

FIG. 7A/B: FIG. 7 shows stability of the dimeric RBD protein in the culture supernatant. FIG. 7A) Protein purification chromatography with IMAC-Ni resin after 4 days of waiting and 7.5% SDS-PAGE gel under non-reducing conditions containing the purified protein fraction: 1. molecular weight marker (kDa); 2. Purified dimeric RBD protein. FIG. 7B) Protein purification chromatogram with IMAC-Ni resin after 5 days of waiting and 7.5% SDS-PAGE gel under non-reducing conditions containing the purified protein fraction: 1. molecular weight marker (kDa); 2. Purified dimeric RBD protein.

FIG. 8: FIG. 8 demonstrates the ELIspot responses of PBMC from convalescent SARS-CoV-2 patients 40 days after symptoms against the pool of 19 CD4+ T cell epitopes.

FIG. 9: FIG. 9 demonstrates the ELIspot responses of PBMC from convalescent SARS-CoV-2 patients 40 days after symptoms against the pool of 26 CD8+ T cell epitopes.

FIG. 10: FIG. 10 shows anti-RBD titers among C57Bl/6 mice immunized with recombinant Wuhan dimeric RBD or RBD+8 synthetic peptides encoding CD4+ T cell epitopes.

FIG. 11: FIG. 11 shows cellular immune responses in mice immunized with dimeric Wuhan RBD or dimeric Wuhan RBD+pooled SARS-CoV-2 dipeptides in response to individual SARS-CoV-2 dipeptides.

FIG. 12: FIG. 12 shows the cellular immune response in mice. The graph shows the measurement of the cellular immune response to the RBD stimulus of different immunization groups: negative control (adjuvant), RBD, RBD+dipeptides.

### DETAILED DESCRIPTION OF THE INVENTION

A series of literature data has shown that the main target of neutralizing antibodies against the SARS-CoV2 virus is directed against the spike protein(S). More specifically, antibodies with high neutralizing capacity are directed against the binding domain (receptor binding domain, RBD) to the ACE2 receptor (angiotensin 2 converting enzyme). Thus, the present invention uses the RBD in a vaccine construct against the Covid-19 infection.

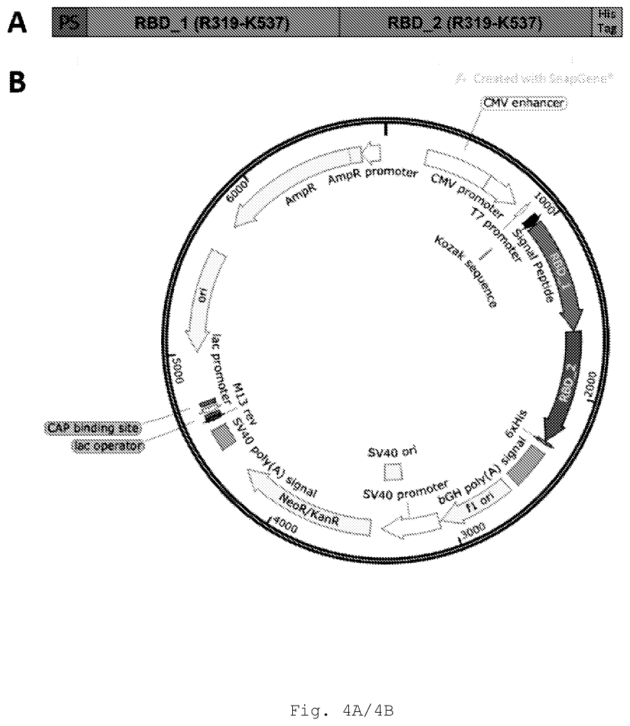

Based on structure-guided design data that showed that a dimeric form of RBD can be more immunogenic than the monomeric form, a dimeric vaccine antigen using the strategy described by Dai et al. (2020) (DAI et al., 2020) is constructed. In this strategy, two sequences of the RBD from the Wuhan strain (amino acids R319 to K537) were drawn in tandem (FIG. 4A/B). To guarantee the expression of the recombinant protein, the signal peptide sequence of the gene that encodes human IgE (YAN et al., 2007) was added and also a sequence of 6 histidines (HisTag), in the C-terminal portion, necessary for purification of vaccine antigen for preclinical testing was (FIG. 4A). This sequence synthesized with codon optimization for expression in mammalian cells and cloned into the expression vector pcDNA3.1 (FIG. 4B) by the company GenScript (Piscataway, USA).

The pcDNA3.1 plasmid containing the dimeric RBD sequence was reconstituted in ultrapure water and transformed by heat shock into chemocompetent Escherichia coli TOP-10 bacteria. As can be seen in FIG. 4B, the pcDNA3.1 plasmid has an ampicillin resistance gene that was used at a concentration of 100 μg/mL for selection of transformed bacteria. Isolated colonies were inoculated in liquid Luria Bertani (LB) medium containing ampicillin and kept under constant agitation (˜300 pm) for 16-18 h at 37° C. Plasmid DNA was extracted using the PureLink HiPure Plasmid Maxiprep Kit (Thermo Fisher Scientific). The quality of the DNA obtained was evaluated by optical spectrophotometry at 260 and 280 nm.

Plasmid DNA was transfected into Chinese hamster ovary cells (ExpiCHO-S) using the ExpiCHOTM Expression System kit (Thermo Fisher Scientific), which contains different media and reagents for carrying out the transfection.

Transient transfection was performed using ExpiFectamine CHO reagent and plasmid DNA, both previously diluted in OptiPRO SFM medium. The bottles were kept under agitation in a humid oven at 37° C. and 8% CO₂ partial pressure. After 16 to 22 h of transfection, the culture received the ExpiCHO Feed medium together with ExpiFectamine CHO Enhancer and the oven temperature was changed to 32° C., reducing the partial pressure of CO₂ to 5% and shaking the same as the previous day. After 10 days of expression, the culture supernatant was clarified by centrifugation and subsequently dialyzed for purification by affinity chromatography on nickel resin (IMAC-Ni, Cytiva) or by ion exchange using the cationic resin S-Sepharose (Cytiva).

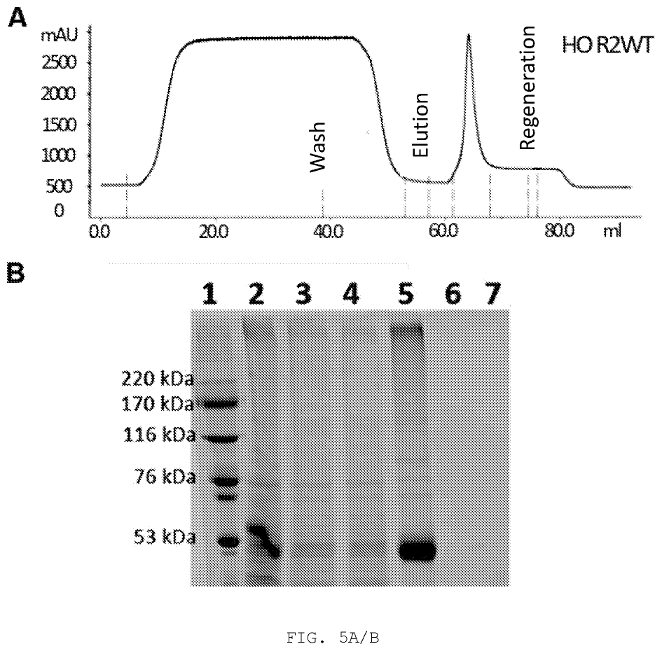

FIG. 5A/B shows the purification chromatogram of the dimeric RBD protein on the IMAC-Ni column (FIG. 5A), as well as a 7.5% SDS-PAGE gel containing different fractions (FIG. 5B). For this purification, the culture supernatant was dialyzed into 20 mM NaH₂PO₄, 500 mM NaCl and 50 mM imidazole buffer (pH 7.4). The IMAC-Ni column was equilibrated with this same buffer and the supernatant containing the dimeric RBD protein was loaded onto the column. Washing was also performed with the same buffer and the protein was eluted in 20 mM NaH₂PO₄, 500 mM NaCl and 300 mM imidazole (pH 7.4). As can be seen in FIG. 5B, the dimeric RBD protein was eluted in elution fraction 1 (column 5). After elution, the eluate was diafiltered and concentrated with Amicon Ultra 10 MWCO (Merck Sigma) into PBS.

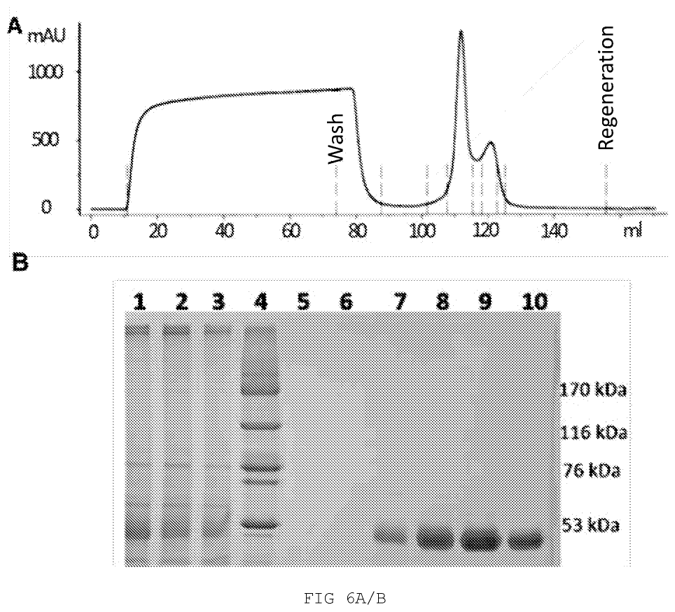

The result of the purification of the dimeric RBD protein by ion exchange is shown in FIG. 6A/B. In this case, the culture supernatant was dialyzed in buffer containing 50 mM Tris-HCl (pH 7.4). This same buffer was used to equilibrate and wash the S-Sepharose column. Elution was performed through a concentration gradient of 0 to 500 mM NaCl (pH 7.4) (FIG. 6A). FIG. 6B shows a 7.5% SDS-PAGE gel containing different fractions where we can see that the dimeric RBD protein was successfully eluted between fractions 3 and 6 (columns 7 to 10). In the same way as performed for the elution of the affinity column, the different fractions containing the protein were mixed, diafiltered and concentrated with Amicon Ultra 10 MWCO (Merck Sigma) for PBS.

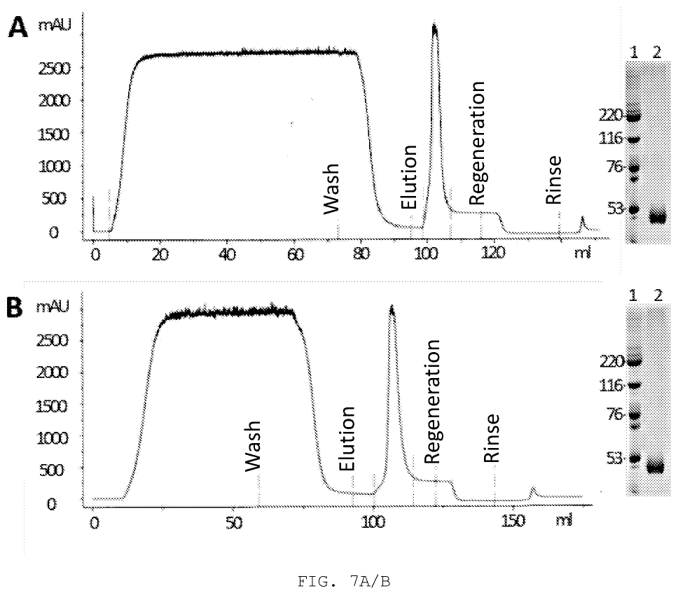

The dimeric RBD protein was further expressed at other times, with consistent results. To test the stability of the protein in the culture supernatant, a transient transfection was performed and then the supernatants were stored for 4 or 5 days at 4° C. before dialysis and purification. After this time, the dimeric RBD protein was purified by nickel resin affinity chromatography, exactly as described above. FIG. 7A/B shows the chromatogram and a 7.5% SDS-PAGE gel containing the purified protein after 4 days (FIG. 7A) and 5 days (FIG. 7B) of waiting.

These results led to the conclusion that the dimeric RBD protein is stable in the culture supernatant for at least 5 days before purification at 4° C.

Thus, the present invention refers to a combination of at least eight synthetic peptides including the sequences below, either simple or having covalent modifications, such as miristoylation and other forms of lipopeptides, added terminal cysteines or other forms that may allow polymerization, referred to herein as epitopes, selected from the entire SARS-CoV-2 proteome sequence (GenBank MN908947.3), which bind in a promiscuous manner multiple HLA-DR molecules and are recognized by CD4+ T-lymphocytes in patients infected by the SARS-CoV-2 virus.

Said epitopes are selected from the group consisting of the sequences of Table 1 below:

TABLE 1
Potential SARS-COV-2 CD4+ T cell epitopes

| SEQ. ID | | Synthetic | | |
|---|---|---|---|---|
| NO. | Protein | Peptides | Start | End |
| SEQ ID | Spike | FGEVFNATRFASVYA | 334 | 352 |
| NO.: 1 | | | | |
| SEQ ID | Spike | GNYNYLYRLFRKSNL | 443 | 466 |
| NO.: 2 | | KPFER | | |
| SEQ ID | Spike | VGYQPYRVVVLSFEL | 503 | 522 |
| NO.: 3 | | LHAPA | | |
| SEQ ID | Spike | IPFAMQMAYRFNGIG | 896 | 915 |
| NO.: 4 | | VTQNV | | |
| SEQ ID | Spike | QLIRAAEIRASANLA | 1009 | 1028 |
| NO.: 5 | | ATK | | |
| SEQ ID | Spike | KAHFPREGVFVSNGT | 1086 | 1105 |
| NO.: 6 | | HWFVT | | |
| SEQ ID | Envelope | SFYVYSRVKNLNSSR | 55 | 72 |
| NO.: 7 | | VPD | | |
| SEQ ID | Membrane | NRFLYIIKLIFLWLL | 43 | 62 |
| NO.: 8 | | WPVTL | | |
| SEQ ID | Membrane | ACFVLAAVYRINWIT | 63 | 81 |
| NO.: 9 | | GGIA | | |
| SEQ ID | Membrane | ASFRLFARTRSMWSF | 98 | 113 |
| NO.: 10 | | N | | |
| SEQ ID | Nucleocapsid | DQIGYYRRATRRIRG | 80 | 97 |
| NO.: 11 | | G | | |
| SEQ ID | Nucleocapsid | ALALLLLDRLNQLES | 212 | 234 |
| NO.: 12 | | KM | | |
| SEQ ID | Nucleocapsid | SAFFGMSRIGMEVTP | 308 | 330 |
| NO.: 13 | | SGTW | | |
| SEQ ID | ORF1 | TSLLVLVQSTQWSLF | 3589 | 3613 |
| NO.: 14 | | | | |
| SEQ ID | ORF3a | SDFVRATATIPIQAS | 26 | 40 |
| NO.: 15 | | | | |
| SEQ ID | ORF3a | INFVRIIMRLWLCWK | 118 | 137 |
| NO.: 16 | | CRSKN | | |
| SEQ ID | ORF7a | AAIVFITLCFTLKRK | 105 | 120 |
| NO.: 17 | | T | | |
| SEQ ID | ORF8 | MKFLVFLGIITTVAA | 1 | 17 |
| NO.: 18 | | FH | | |
| SEQ ID | ORF8 | SKWYIRVGARKSAPL | 43 | 57 |
| NO.: 19 | | | | |

Moreover, said epitopes are selected from the group consisting of the sequences of Table 1, particularly, from the group consisting of: SEQ ID NO: 4; SEQ ID NO: 5; SEQ ID NO: 11; SEQ ID NO: 12; SEQ ID NO: 13; SEQ ID NO: 14 and SEQ ID NO: 16.

The selected epitopes were those predicted to bind over the chosen threshold (3%) to the greatest possible number of HLA-DR molecules in a “promiscuous” manner (sequences predicted to bind to at least 26 of the 51 HLA-DR molecules available in the algorithm with a threshold of 3%, thus selecting epitopes with a high chance of binding HLA-DR molecules with great avidity).

To attain the final peptide sequences of Table 1, N- and C-terminal flanking residues were added, so as to increase the percentage of individuals recognizing each peptide. The combination of synthetic peptides was designed to elicit or detect SARS-CoV-2 specific responses of over 99% of the world population according to HLA frequency data in the iedb.org server. Each HLA-DR molecule was predicted to bind to at least 10 distinct peptides according to the in-silico analysis using https://webs.iiitd.edu.in/raghava/propred/.

According to the in-silico analysis, an increasing number of epitopes would allow recognition of a higher number of peptides per HLA-DRB1 and per individual, reaching a minimum of 10 peptides/HLA-DRB1 using the complete set of peptides from SEQ ID NO: 1 to 19. An increasing number of epitopes/individual increases the amplitude of the vaccination. The more peptides each vaccinee recognizes, the more difficult it is that mutations could jeopardize vaccine-induced protection to new mutant viruses. Complete coverage with ample breadth (number of T cell epitopes recognized per individual) is an expected emergent property of a combination of promiscuous HLA-binding epitopes. Each epitope individually will never be as antigenic/immunogenic in a genetically heterogeneous population as a combination of said individual epitopes.

Moreover, the epitopes of the present invention used in a combination are particularly derived from the spike protein, envelope protein, membrane protein, nucleocapsid protein, and other SARS-CoV-2 ORFs.

One of the advantages of the present invention is the recognition of said combination of epitopes by CD4+ T-cells, an emergent property of the combination of promiscuous epitopes capable of binding to multiple HLA-DR molecules. Another advantage is that it targets many non-Spike viral proteins which are less prone to antibody-induced immune pressure and mutations.

According to the present invention, “epitopes” mean the epitopes mentioned above, their functional equivalents and mimetic sequences thereof.

A “functional equivalent” refers to structurally distinct sequences, fragments, analogues, derivatives or associations, which perform the same function to achieve equal results. It is understood that any alterations made by those skilled in the art, which lead in an obvious manner to equivalent effects, shall also be considered as a part of the invention. More particularly, functional equivalents are the sequences presenting homology of at least 12 amino acids to the epitopes described above and perform the same function of said epitopes, exhibiting equal or similar results.

In accordance with the present invention, “mimetic sequences” are understood as being non-natural amino acid sequences with modified structures, so that they present functions and results equal or similar to the sequences of the epitopes of the present invention.

The above mentioned epitopes are putative immunodominant SARS-CoV-2 CD4+ T cell epitopes. Said epitopes were selected from the entire SARS-CoV-2 proteome sequence (GenBank MN908947.3). Such synthetic peptides were designed to bind to at least 50% of the 51 HLA-DR molecules in the bioinformatic server https://webs.iiitd.edu.in/raghava/propred/.

Particularly, the combination of eight or more epitopes of the present invention comprises the SEQ ID NOS of the Table 2 below and may allow ample coverage. The in silico studies suggested that with at least the following 10 peptides, one would detect T cell responses against at least 5 peptides in all individuals.

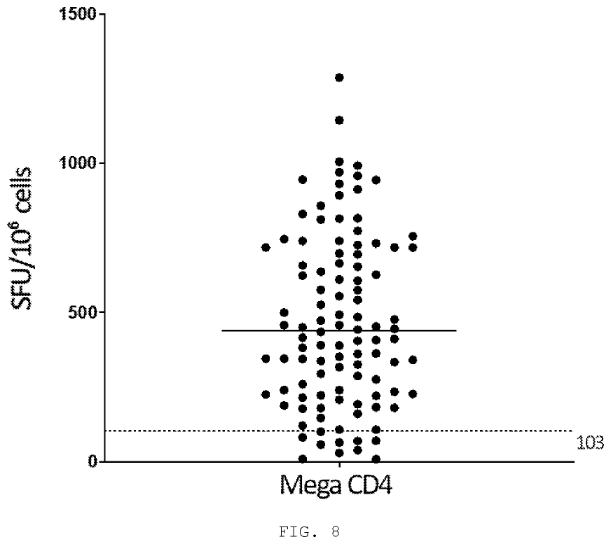

In order to evaluate the capacity of the selected combination of at least eight epitopes to be recognized by T-lymphocytes of individuals, said epitopes (described in Table 1) were synthesized in solid phase by using Fmoc chemistry and having a C-terminal amide. Synthetic peptide stocks were diluted to 5 mg/mL in dimethylsulphoxide, followed by ELISPOT assays for detection of IFN-γ-producing cells in response to the epitopes using peripheral blood mononuclear cells (PBMC) in 98 COVID-19 convalescent patients (mild disease cases not requiring hospitalization) at least two months after signs of infection (FIG. 8). Synthetic peptides were diluted to make a pool of 5 mg/mL and added to wells (total peptide concentration 5 μg/mL, individual concentration 0,25 μg/ml). The cryopreserved cells were maintained in culture for 16 hours, washed and placed on ELISPOT plates in the presence of the epitopes, incubated for a further 18 hours. ELISPOT plates were developed for the identification of spot-forming cells/Interferon-γ-producing cells (IFN-γ SFC), which were then counted in an automated counter (Zeiss KS ELISpot/Axioplan 2). PBMC samples from seventeen seronegative control individuals obtained prior to the COVID-19 pandemic were used to calculate the background.

Responses in no-peptide background wells were subtracted from peptide-elicited in each of the 98 tested individuals, and the results were plotted on the graph of FIG. 8. The response cutoff (103 Spot-forming cells (SFC)/10⁶ PBMC) was calculated as the average plus 3 standard deviations of the ELISPOT responses to the 20 synthetic peptides as mentioned above in Table 1. Individuals with responses above the cutoff were considered as possessing a T cell response to the SARS-CoV-2 peptides, which indicates 97% recognition.

The combination of all epitopes of the present invention have further use in diagnostic methods and in trials for the evaluation of the immune response of CD4+ T-lymphocytes against the SARS-CoV-2 virus. A diagnostic method, for instance, to detect or monitor cellular response to infection, or vaccination, allowing to identify breakthrough infection in individuals immunized with SARS-CoV-2 subunit vaccines, in vivo, ex vivo or in vitro. Another diagnostic method, for example, in vitro cellular immune response assay strategy for diagnosis in large quantities (scalable r hundreds of reactions/day) is also an embodiment of the present invention. Another example would be their use in in vivo diagnosis when a response is observed after administration of said peptides.

Furthermore, the combination of at least eight epitopes of the present invention are useful for the preparation of vaccines, to provide T cell help and increase of the immunogenicity and protective properties thereof. Said vaccines may be more effective than those already known in the state of the art since the combination of at least eight epitopes of the present invention are recognized by the T-cells in a majority of individuals, thus covering a significant proportion of the population exposed to the virus.

It is also an object of the present invention a composition comprising the combination of at least eight of the epitopes of SEQ ID NO:1 to SEQ ID NO: 19. Said composition further comprises a pharmaceutically acceptable carrier or vehicle.

The compositions of the present invention may be in the solid or liquid form. Said compositions may be formulated for a rapid or prolonged release of their components and may further comprise compounds for stimulating and/or inhibiting the immunologic system. Said compositions may be prepared in accordance with conventional methods already known in the state of the art.

Further objects of the present invention are uses of the composition described above. This includes the use of said composition in the preparation of vaccines, in diagnostic methods and tests for evaluating the immune response of CD4+ T-lymphocytes against SARS-CoV-2 virus, as described above for the mentioned combination of at least eight epitopes as the ones set forth in SEQ ID NO:1 to SEQ ID NO: 19 of Table 1 above.

The present invention also refers to a vaccine construct, wherein said vaccine construct comprises CD4+ T cell epitopes in the form of synthetic peptides in combination with RBD monomers or dimers.

The vaccine construct of the present invention comprises a recombinant protein conformed by a single polypeptide chain formed by connecting one to three novel betacoronavirus (SARS-CoV-2) Spike(S) protein receptor binding domain (RBD) in tandem and/or through a linker to peptide string conformed by CD4+ epitopes or CD4+ plus CD8+ T cell epitopes.

The CD8+ T cell epitopes are selected from the group consisting of the sequences of Table 2 below:

TABLE 2
SARS-COV-2 CD8+ T cell epitopes

| | | | | SEQ ID |
|---|---|---|---|---|
| Protein | Start | End | Sequence | NO: |
| Spike | 89 | 97 | GVYFASTEK | SEQ ID |
| | | | | NO.: 20 |
| Spike | 269 | 277 | YLQPRTFLL | SEQ ID |
| | | | | NO.: 21 |
| Spike | 269 | 277 | MIAQYTSAL | SEQ ID |
| | | | | NO.: 22 |
| Spike | 691 | 699 | SIIAYTMSL | SEQ ID |
| | | | | NO.: 23 |
| Spike | 1220 | 1228 | FIAGLIAIV | SEQ ID |
| | | | | NO.: 24 |
| Nucleocapsid | 307 | 315 | FAPSASAFF | SEQ ID |
| | | | | NO.: 25 |
| Nucleocapsid | 219 | 227 | LALLLLDRL | SEQ ID |
| | | | | NO.: 26 |
| Nucleocapsid | 222 | 230 | LLLDRLNQL | SEQ ID |
| | | | | NO.: 27 |
| Membrane | 171 | 179 | ATSRTLSYY | SEQ ID |
| | | | | NO.: 28 |
| Membrane | 61 | 70 | TLACFVLAAV | SEQ ID |
| | | | | NO.: 29 |
| NSP 3 | 1081 | 1089 | YYKKDNSYF | SEQ ID |
| | | | | NO.: 30 |
| NSP 3 | 1374 | 1382 | ASMPTTIAK | SEQ ID |
| | | | | NO.: 31 |
| NSP 3 | 1802 | 1810 | AELAKNVSL | SEQ ID |
| | | | | NO.: 32 |
| NSP 3 | 686 | 694 | TISLAGSYK | SEQ ID |
| | | | | NO.: 33 |
| NSP 3 | 887 | 895 | GEAANFCAL | SEQ ID |
| | | | | NO.: 34 |
| NSP 5 | 219 | 227 | FLNRFTTTL | SEQ ID |
| | | | | NO.: 35 |
| NSP 6 | 84 | 92 | VYMPASWVM | SEQ ID |
| | | | | NO.: 36 |
| NSP 9 | 23 | 31 | CTDDNALAY | SEQ ID |
| | | | | NO.: 37 |
| RNA | 253 | 261 | AESHVDTDL | SEQ ID |
| polymerase | | | | NO.: 38 |
| RNA | 500 | 508 | KSAGFPENK | SEQ ID |
| polymerase | | | | NO.: 39 |
| RNA | 907 | 915 | LINDNTSRY | SEQ ID |
| polymerase | | | | NO.: 40 |
| exonuclease | 223 | 231 | TYACWHHSI | SEQ ID |
| | | | | NO.: 41 |
| exonuclease | 232 | 240 | GFDYVYNPF | SEQ ID |
| | | | | NO.: 42 |
| exonuclease | 288 | 296 | KRVDWTIEY | SEQ ID |
| | | | | NO.: 43 |
| exonuclease | 487 | 495 | HANEYRLYL | SEQ ID |
| | | | | NO.: 44 |
| Helicase | 386 | 394 | VVNARLRAK | SEQ ID |
| | | | | NO.: 45 |

The vaccine construct of the present invention is described by the following technical scheme:

The amino acid sequence of the single polypeptide chain is represented as:
(A) RBD1-RBD2 or RBD1-RBD2-RBD3 as shown in SEQ ID NO: 54, 55, 56 and 57;
(B) RBD1-linker-Peptide String as shown in SEQ ID NO: 58, 59 and 60;
(C) RBD1-RBD2-linker-Peptide String as shown in SEQ ID NO: 61, 62, 63, 64, 65, 66 and 67;
(D) RBD1-linker-RBD2 as shown in SEQ ID NO: 68 and 69;
(E) RBD1-linker-Peptide String-linker-RBD2 as shown in SEQ ID NO: 70, 71, 72, 73, 74, 75, 76 and 77, wherein,
(1) the amino acid sequence of the novel betacoronavirus (SARS-CoV-2) Spike(S) protein receptor binding domain (RBD) comprises the residues 319-537 (SEQ ID NO: 54 to 67, SEQ ID NO: 70 to 75) and 330-528 (SEQ ID NO: 68, 69, 76 and 77) of the S protein;
(2) RBD1, RBD2 and RBD3 represent the amino acid sequences of the novel betacoronavirus (SARS-CoV-2) Spike(S) protein receptor binding domain (RBD) from the original Wuhan strain and their substitutions corresponding variant (B.1.1.529, K417N, E484K and N501Y), Gamma variant (P1, K417T, E484K and N501Y) Delta variant (B.1.617.2, L452R and T478K) and Omicron (B.1.1.159, BA1, G339D, S371L, S373P, S375F, K417N, N440K, G446S, S477N, T478K, E484A, Q493R, G496S, Q498R, N501Y and Y505H);
(3) The novel betacoronavirus (SARS-CoV-2) Spike(S) protein receptor binding domain (RBD) and the peptide strings are connected in tandem (SEQ ID NO: 54, 55, 56 and 57) and/or through the peptide linkers (SEQ ID NO:58 to 77). Specifically, the novel betacoronavirus (SARS-CoV-2) Spike(S) protein receptor binding domain (RBD) and the peptide strings are connected by EAAAKEAAAKEAAAK (SEQ ID NO: 58 to 67), KPKPKP (SEQ ID NO: 70, 72, 74 and 75), GGGGS (SEQ ID NO: 71 and 73) or PKPK (SEQ ID NO: 68, 69, 76 and 77) sequences;
(4) The peptides strings are constructed by CD4+ and/or CD8+ T cell epitopes (see CD4+ and CD8+ T cell epitope lists, SEQ ID NO: 1 to 45) connected through the GPGPG linker. Using the CD4+ and CD8+ T cell epitopes list were constructed dipeptides linked by GPGPG sequence (SEQ ID NO: 46-53), CD4+ T cell epitopes string 1-4 (SEQ ID NO: 54), CD4+ T cell epitopes string 5-8 (SEQ ID NO: 55), CD4+ T cell epitopes string 08 (SEQ ID NO: 56), CD8+ T cell epitopes string 11 (SEQ ID NO: 57) and CD4+ and CD8+ T cell epitopes string 22 (SEQ ID NO: 58).

In addition, is also an object of the present invention the vaccine construct wherein RBD1, RBD2 and RBD3 of item (2) above represent the amino acid sequences of the betacoronavirus (SARS-CoV-2) Spike(S) protein receptor binding domain (RBD) from any new variants of concern.

Said vaccine construct comprises the combination of at least eight of the epitopes described above in Table 1, in association with one or more pharmaceutically acceptable adjuvants, vehicles, excipients, binding agents, carriers or preservatives.

The vaccine construct in accordance with the present invention may be formulated according—but not restricted to—the following forms, including the combination of eight or more of the epitopes as set forth in SEQ ID NO:1 to SEQ ID NO: 19 as described in Table 1 above:
a) combining the eight or more epitopes as set forth in SEQ ID NO: 1 to SEQ ID NO: 19 with adjuvants; or
b) a recombinant or synthetic DNA or RNA construction with the sequences of eight or more of the new epitopes, particularly containing other protein products; or
c) a recombinant protein or synthetic peptide construction with sequences of the new epitopes, particularly combined with adjuvants; or
d) a viral vector containing sequences of the new epitopes; or
e) a virus-like particle where epitopes are expressed in encoded proteins; or
f) a combination of the new epitopes with SARS-CoV-2 epitopes and immunogens already known in the state of the art, or as a booster to such known SARS-CoV-2 immunogens.

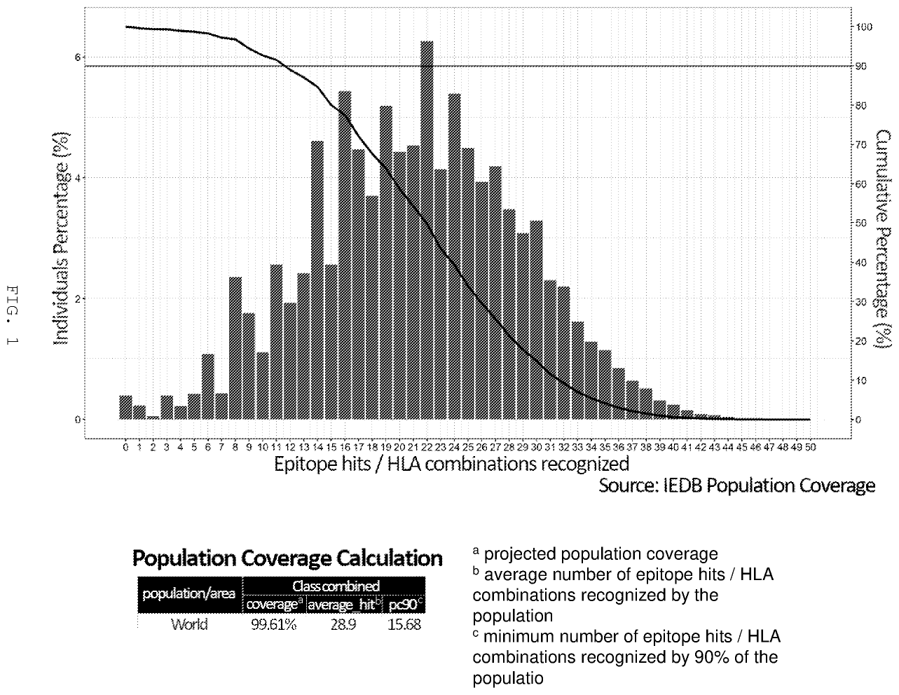

Another object of the present invention is a method for the identification of the new epitopes for the combination of the present invention that allow high coverage of the world population (FIG. 1), which comprises the steps of:
a) selecting of the peptides from the entire SARS-CoV-2 proteome sequence, wherein said peptides bind to at least 50% of the HLA-DR molecules among in the bioinformatic server https://webs.iiitd.edu.in/raghava/propred/;
b) expanding of the sequences of said peptides selected at the N-terminal and C-terminal ends;
c) performing world population coverage studies based on frequencies of HLA molecules predicted to bind to peptides at the iedb.org website;
d) synthesizing of corresponding peptides;
e) confirming and testing for SARS-CoV-2-specific responses in sensitized individuals that have had mild COVID-19 disease (no hospitalization) and have recovered well.

It was also performed in silico population coverage analysis of selected combinations of 8 and 11 CD4+peptides present in some of the constructions mentioned along this document. The world population coverage of a combination of 11 selected CD4+peptides which are part of some of the constructions mentioned above, also assessed at the IEDB population coverage at iedb.org indicate that 90.6% of the world population could recognize at least one of the epitopes, and on average each individual would recognize 9.8 HLA/epitope combinations. The world population coverage of one combination of 8 CD4+ T cell epitopes that are part of some of the constructions mentioned above, also would cover 90.6% of the population, on average each individual recognizing 9.5 HLA/epitope combinations The world population coverage of one combination of 8 CD4+ T cell epitopes that are part of some of the constructions mentioned above, also would cover 90.6% of the population, on average each individual recognizing 6.3 HLA/epitope combinations. Altogether these in silico results indicate that subsets of 8 or 11 CD4+epitopes attain high population coverage with a high number of peptides capable of binding per HLA molecule.

The following examples are illustrative and provide a clearer and more consistent understanding of the invention but are not intended to limit its scope.

### EXAMPLES

### Example 1

### T Cell Responses to the Selected CD4+ and CD8+ T Cell Epitopes Among SARS-CoV-2 Convalescent Patients

To evaluate the cellular response to predicted T cell epitopes, samples from 110 convalescent participants, collected approximately 40 days after infection, were used. Blood was processed to obtain peripheral blood mononuclear cells (PBMC), which were cryopreserved in liquid nitrogen until use.

After in silico analysis of the SARS-CoV-2 sequence and selection of potentially more promiscuous CD4+ T cell epitopes, the selected peptides were synthesized. In total, the synthesis consisted of 19 peptides from CD4+ T cells and 26 peptides from CD8+ T cells. Synthetic peptides were used to stimulate PBMC for 24 h and analyzed by ELISPOT to assess IFN-g production.

Analysis showed that the synthetic peptides corresponding to the CD4+ T cell epitopes have ample immune convalescent coverage among subjects, inducing a T lymphocyte response in 92% of the samples tested (FIG. 8).

Then, to analyze the overall immunodominance of the CD4+ T cell epitopes, the PBMCs were stimulated with each peptide individually. Again, the magnitude of the response differed between subjects, however, each peptide alone induced IFN-g-producing T cells in 60%-80% of the patients tested. T cells from each patient recognized on average 14 of the 19 CD4+ T cell peptides, or 74% of the epitopes tested and 93% of patients recognized at least one CD4+ T cell epitope (data not shown), an indication of the promiscuity of HLA class II binding and T cell responses.

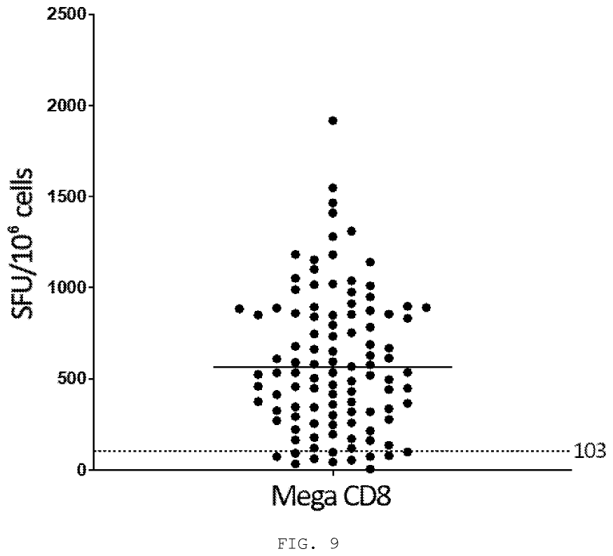

Similar analyses of T cell recognition of the 26 CD8+ T cell epitopes were performed in the same convalescent cohort. It was observed that the 26 synthetic peptides corresponding to the CD8+ T cell epitopes have ample immune a T coverage among convalescent subjects, inducing lymphocyte response in 91% of the samples tested (FIG. 9).

To analyze the overall immunodominance of the CD8+ T cell epitopes, the PBMCs were stimulated with each CD8+peptide epitope individually. The magnitude of the response differed between subjects, however, T cells from each patient recognized on average 20 of the 26 CD4+ T cell peptides, or 77% of the epitopes tested and 95% of patients recognized at least one CD4+ T cell epitope (data not shown), indicating a surprising promiscuity of HLA class I binding and T cell responses.

The results indicate that the peptides predicted in silico were highly recognized by cells from convalescent patients and there was no pattern of immunodominance in response to peptides derived from different regions of the virus.

### Example 2—Humoral Response to a Vaccine Encoding RBD and Synthetic Peptides: Induction of Specific Antibodies

In order to identify the best vaccine candidate and immunization strategy, C57BL/6 mice were immunized as described in Table 3 below. The animals used came from the Center for the Development of Experimental Models for Medicine and Biology (CEDEME) and were kept in free of pathogens in the vivarium of the Discipline of Immunology (DMIP/UNIFESP), with a controlled light-dark cycle (12:12) and free access to water and feed. This project was previously approved by the Ethics Committee for the Use of Animals (CEUA-UNIFESP) under number 4813280820.

TABLE 3
Immunization regimen for humoral and cellular immune responses

| | # of | Immunization | Antigen | 3 Doses |
|---|---|---|---|---|
| Groups | animals | route | dose | (2 week apart) |
| 1 | 3 | SC | — | AS03 |
| 2 | 4 | SC | 5 μg | RBD + AS03 |
| 3 | 4 | SC | 5 μg + | RBD + Dipep. + |
| | | | 50 μg | AS03 |

The animals were immunized with 5 μg of 5 μg of Wuhan dimeric RBD protein or 5 μg of Wuhan dimeric RBD protein plus 50 μg of eight synthetic dipeptides as set forth in SEQ ID NO: 46 to 53, each encompassing one CD4+ and one CD8+ T cell epitope as mentioned in Table 4 below, and in the presence of AS03 adjuvant (1:1 v/v) subcutaneously (SC), 100 μL with an interval of 15 days between doses. Control groups received only the ASO3 adjuvant via the SC route.

TABLE 4
Eight synthetic dipeptides containing each
one CD4+ and one CD8+ T cell epitope.

| | | | SEQ | | |
|---|---|---|---|---|---|
| | | | ID | below: K + CD4 + | |
| DIPEPTIDES | | | NO: | GPGPG + CD8-NH2 | length |
| P1 | M1- | 1 + | SEQ | KACFVLAAVYRINWITGG | 34 |
| | NC1 | G | ID | IAGPGPGKPRQKRTAT | |
| | | | NO: | | |
| | | | 46 | | |
| P2 | M2- | 2 + | SEQ | KASFRLFARTRSMWSFNV | 31 |
| | NC2 | H | ID | GPGPGLALLLLDRL | |
| | | | NO: | | |
| | | | 47 | | |
| P3 | M3- | 6 + | SEQ | KNRFLYIIKLIFLWLLWP | 35 |
| | S1 | E | ID | VTLKGPGPGGVYFASTEK | |
| | | | NO: | | |
| | | | 48 | | |
| P4 | O7a- | 8 + | SEQ | KAAIVFITLCFTLKRKTG | 31 |
| | RNAP2 | F | ID | PGPGKSAGFPFNK | |
| | | | NO: | | |
| | | | 49 | | |
| P5 | S3- | 7 + | SEQ | KIPFAMQMAYRFNGIGVT | 35 |
| | NSP5 | D | ID | QNVGPGPGFLNRFTTTL | |
| | | | NO: | | |
| | | | 50 | | |
| P6 | S2- | 5 + | SEQ | KQLIRAAEIRASANLAAT | 32 |
| | EXO | B | ID | GPGPGHANEYRLYL | |
| | | | NO: | | |
| | | | 51 | | |
| P7 | O8- | 3 + | SEQ | KMKFLVFLGIITTVAAFH | 32 |
| | NSP3 | A | ID | GPGPGYYKKDNSYF | |
| | | | NO: | | |
| | | | 52 | | |
| P8 | O3a- | 4 + | SEQ | KINFVRIIMRLWLCWKCR | 35 |
| | RNAP | C | ID | SKNGPGPGLTNDNTSRY | |
| | | | NO: | | |
| | | | 53 | | |

For analysis of the humoral immune response, the animals' blood was collected 14 days after each dose and the titers of dimeric-specific RBD antibodies were determined by ELISA assay. For this, ELISA plates (High binding, Costar) were used and 250 ng of the dimeric RBD recombinant protein produced and characterized as described in the previous items were added to each well. After incubation for 16 to 18 h at room temperature, the plates were washed with 0.02% PBS-Tween 20 (PBS-T0.02) and blocked (PBS-T0.02/1% BSA) for 1 hour at room temperature. After 2 hours of incubation with serial dilutions of the serum of the immunized mice, the contents of the wells were discarded, and the plates washed with PBS-T 0.02. The secondary antibody (anti-mouse IgG linked to peroxidase-KPL) diluted in PBS-T0.02/BSA 1% in the proportion of 1:10,000 was added and incubated for another 2 hours at room temperature. After three more washes with PBS-T 0.02, the revelation was made with a solution containing OPD (Sigma) dissolved in a solution containing 0.2 M NaH₂PO₄ and 0.1 M citric acid (pH 4.7) plus 10 μL of 30% H₂O₂. After 15 minutes, the reaction was stopped with 50 μL of a 4N solution of H₂SO₄. Subsequently, the plates were analyzed in a 96-well plate reader, at a wavelength of 492 nm (PerkinElmer).

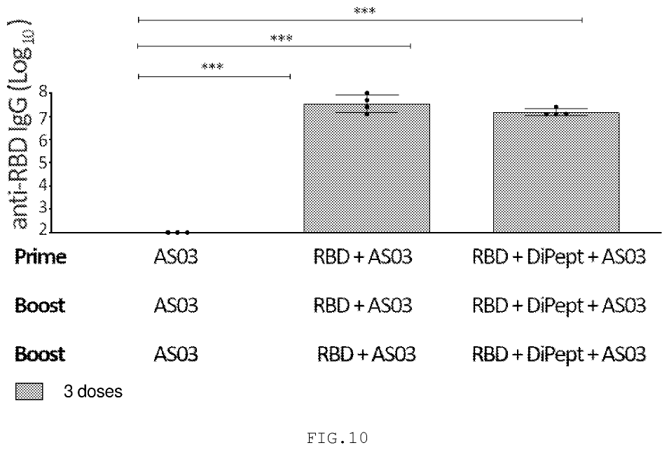

Analysis of serum antibody production showed that the magnitude of IgG production was comparable among the animals that received RBD or RBD+8 synthetic peptides encoding SARS-CoV-2 CD4+ and CD8+ T cell epitopes (FIG. 10).

### Example 3—Cellular Immune Response to a Vaccine Encoding SARS-CoV-2 RBD and Selected SARS-CoV-2 Synthetic Peptides: Induction of Strong Peptide-Specific T Cell Responses

In addition to the humoral immune response, the cellular immune response plays an important role in protection against SARS-CoV-2. To assess the differentiation of T lymphocytes specific for the recombinant SARS-CoV-2 RBD antigens in mice immunized with 5 μg of Wuhan dimeric RBD protein or 5 μg of Wuhan dimeric RBD protein plus 50 μg of a pool of eight synthetic dipeptides as set forth in SEQ ID NO: 46 to 53, each encompassing one CD4+ T cell epitope and one CD8+ T cell epitope (see Table 4 above in Example 2).

The profile of IFN-γ producing cells by ELIspot in the spleen of immunized mice was analyzed. Therefore, after euthanasia, the spleen of each animal was collected with the aid of surgical material, in laminar flow. Cells were obtained and washed by centrifugation with 10 ml of supplemented RPMI medium (Gibco). Then, the cells were treated with ACK hemolytic buffer (0.15M NH₄Cl, 1 mM KHCO₃, 0.1 mM Na₂EDTA) and then washed twice with 10 ml RPMI medium. At the end, the cells were resuspended in 1 ml of R10 medium (supplemented RPMI medium, containing 10% fetal bovine serum—Gibco).

The ELISpot assay was performed using the Mouse IFN-γ ELISpot kit (BD Biosciences) according to the manufacturer's instructions. Briefly, the capture antibody (purified IFN-γ anti-mouse) was added to the plate (Millipore Multi-Screen IP-MAIPS 4510) at a concentration of 5 μg/mL in a final volume of 100 μL and the plate was stored overnight at 4° C. Then, the wells were washed with PBS and blocked with RPMI medium containing 10% fetal bovine serum (R10) for 2 hours at room temperature. After this period, stimulus (each individual dipeptide, 5 μg/mL) and cell suspensions were added. The plate was incubated at 37° C. in an oven containing 5% CO₂ overnight. Subsequently, the plates were washed with deionized water and with PBS-0.05% Tween20 (PBS-T). Detection antibody (biotinylated anti-IFN-γ) was diluted in PBS-10% FBS and added to the wells at a final concentration of 2 μg/mL and the plate was incubated for 2 h at room temperature. After washing the wells 3× with PBS-T, the enzyme conjugate (streptavidin-peroxidase) was diluted in PBS-10% FBS and added to the wells (100 μL/well), followed by a further 1h incubation step at room temperature. For development, the wells were washed 4× with PBS-T and then 2× with 1×PBS. 100 μof AEC (3-amino-9-ethylcarbazole-BD) solution was then added per well and spot formation was monitored. The reaction was stopped with 5 washes of the plate with deionized water. The number of UFS (Spot Forming Units) was performed on the reader AID ELISpot Reader System (Autoimmun Diagnostika GmbH, Germany).

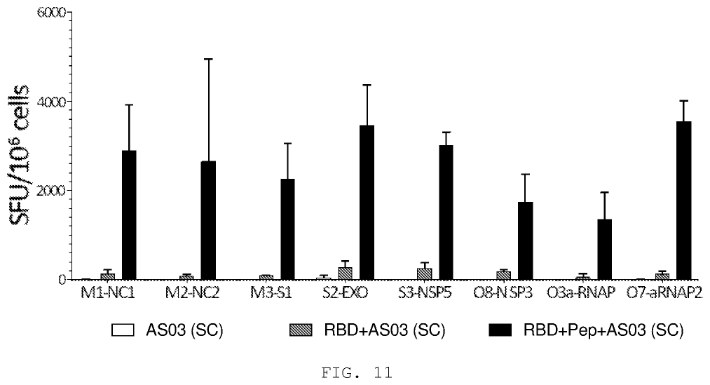

FIG. 11 shows the cellular immune responses to each individual dipeptide among mice immunized with the Wuhan dimeric RBD protein or the dimeric RBD protein+8 dipeptides. It can be seen that all peptides elicited powerful immune responses, above 1,000 SFU/10⁶ cells in the group coimmunized with synthetic peptides. This indicates that the selected SARS-CoV-2 peptides are highly immunogenic.

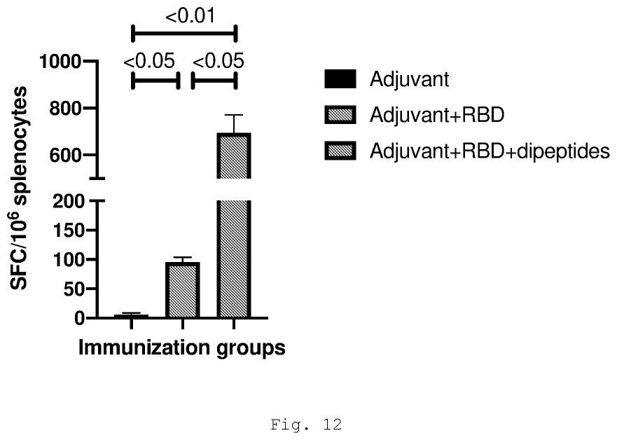

Further results indicating an additional non-cognate effect in the cellular immune response when the dimeric RBD recombinant protein+8 dipeptides as set forth in SEQ ID NO: 46 to 53 were used are shown in FIG. 12.

It is possible to identify the increase of the cellular immune response of the immunization group using adjuvant+RBD+dipeptides, said increase being directly caused by an additional immunization due to the dipeptides as set forth in SEQ ID NO: 46 to 53, which do not have epitopes in common with the RBD protein.

### BIBLIOGRAPHIC REFERENCES

DAI, Lianpan et al. A Universal Design of Betacoronavirus Vaccines against COVID-19, MERS, and SARS. Cell, v. 182, n. 3, p. 722-733.el1, ago. 2020.
SCHMIDT, Fabian et al. Measuring SARS-CoV-2 neutralizing antibody activity using pseudotyped and chimeric viruses. Journal of Experimental Medicine, v. 217, n. 11, 2 Nov. 2020.
YAN, Jian et al. Enhanced Cellular Immune Responses Elicited by an Engineered HIV-1 Subtype B Consensus-based Envelope DNA Vaccine. Molecular Therapy, v. 15, n. 2, p. 411-421, fev. 2007.

1-14. (canceled)

15. A combination of epitopes wherein said combination comprises at least eight T cell epitopes from the SARS-CoV-2.

16. A combination of epitopes according to claim 15, wherein said epitopes are widely recognized by CD4+ T-lymphocytes.

17. A combination of epitopes according to claim 15, wherein the T cell epitopes are selected from the group consisting of SEQ ID NO: 1 to SEQ ID NO: 19.

18. A combination of epitopes according to claim 17, wherein the T cell epitopes are selected from the group consisting of: SEQ ID NO:4; SEQ ID NO:5; SEQ ID NO: 11; SEQ ID NO: 12; SEQ ID NO:13; SEQ ID NO:14 and SEQ ID NO:16.

19. A vaccine construct comprising:
a. CD4+ T cell epitopes or CD4+ plus CD8+ T cell epitopes, in the form of synthetic peptides, in combination with RBD monomers or dimers; or
b. combining the eight or more epitopes as set forth in SEQ ID NO: 1 to SEQ ID NO:19 with adjuvants; or
c. a recombinant or synthetic DNA or RNA construction with the sequences of eight or more of the new epitopes, particularly containing other protein products; or
d. a recombinant protein or synthetic peptide construction with sequences of the new epitopes, particularly combined with adjuvants; or
e. a viral vector containing sequences of the new epitopes; or
f. a virus-like particle where epitopes are expressed in encoded proteins; or
g. a combination of the new epitopes with SARS-CoV-2 epitopes and immunogens already known in the state of the art, or as a booster to such known SARS-CoV-2 immunogens.

20. A vaccine construct according to claim 19, comprising an amino acid sequence of a single polypeptide chain as represented as
(A) RBD1-RBD2 or RBD1-RBD2-RBD3 as shown in SEQ ID NO: 54, 55, 56 and 57;
(B) RBD1-linker-Peptide String as shown in SEQ ID NO: 58, 59 and 60;
(C) RBD1-RBD2-linker-Peptide String as shown in SEQ ID NO: 61, 62, 63, 64, 65, 66 and 67;
(D) RBD1-linker-RBD2 as shown in SEQ ID NO: 68 and 69;
(E) RBD1-linker-Peptide String-linker-RBD2 as shown in SEQ ID NO: 70, 71, 72, 73, 74, 75, 76 and 77;
wherein,
(1) the amino acid sequence of the novel betacoronavirus (SARS-CoV-2) Spike(S) protein receptor binding domain (RBD) comprises the residues 319-537 (SEQ ID NO: 54 to 67, SEQ ID NO: 70 to 75) and 330-528 (SEQ ID NO: 68, 69, 76 and 77) of the S protein;
(2) RBD1, RBD2 and RBD3 represent the amino acid sequences of the novel betacoronavirus (SARS-CoV-2) Spike(S) protein receptor binding domain (RBD) from the original Wuhan strain and their substitutions corresponding to Beta variant (B.1.1.529, K417N, E484K and N501Y), Gamma variant (P1, K417T, E484K and N501Y) Delta variant (B.1.617.2, L452R and T478K) and Omicron (B.1.1.159, BA1, G339D, S371L, S373P, S375F, K417N, N440K, G446S, S477N, T478K, E484A, Q493R, G496S, Q498R, N501Y and Y505H);
(3) the novel betacoronavirus (SARS-CoV-2) Spike(S) protein receptor binding domain (RBD) and the peptide strings are connected in tandem (SEQ ID NO: 54, 55, 56 and 57) and/or through the peptide linkers (SEQ ID NO: 58 to 77);
(4) the peptide strings are constructed by CD4+ and/or CD8+ T cell epitopes (see CD4+ and CD8+ T cell epitope lists, SEQ ID NO: 1 to 45) connected through the GPGPG linker; wherein from the CD4+ and CD8+ T cell epitopes list were constructed dipeptides linked by GPGPG sequence (SEQ ID NO: 46 to 53), CD4+ T cell epitopes string 1-4 (SEQ ID NO: 54), CD4+ T cell epitopes string 5-8 (SEQ ID NO: 55), CD4+ T cell epitopes string 08 (SEQ ID NO: 56), CD8+ T cell epitopes string 11 (SEQ ID NO: 57) and CD4+ and CD8+ T cell epitopes string 22 (SEQ ID NO: 58).

21. A vaccine construct according to claim 20, wherein the novel betacoronavirus (SARS-CoV-2) Spike(S) protein receptor binding domain (RBD) and the peptide strings are connected by EAAAKEAAAKEAAAK (SEQ ID NO: 58 to 67), KPKPKP (SEQ ID NO: 70, 72, 74 and 75), GGGGS (SEQ ID NO: 71 and 73) or PKPK (SEQ ID NO: 68, 69, 76 and 77) sequences.

22. A vaccine construct of claim 20, wherein RBD1, RBD2 and RBD3 of item (2) represent the amino acid sequences of the betacoronavirus (SARS-CoV-2) Spike(S) protein receptor binding domain (RBD) from any new variants of concern.

23. A vaccine construct according to claim 21, wherein the peptides in combination are set forth in SEQ ID NO: 46 to 53.

24. A vaccine construct according to claim 19, wherein said vaccine construct further comprises one or more pharmaceutically acceptable adjuvants, vehicles, excipients, binding agents, carriers or preservatives.

25. A method of inducing an immune response comprising administering the vaccine construct as defined in claim 19.

26. A method for the identification of SARS-CoV-2 T cell epitopes for a vaccine, comprising the steps of:
a. selecting the peptides from the entire SARS-CoV-2 proteome sequence, wherein said peptides bind to at least 50% of the HLA-DR molecules among in the bioinformatic server https://webs.iiitd.edu.in/raghava/propred/;
b. expanding the sequences of said peptides selected at the N-terminal and C-terminal ends;
c. performing world population coverage studies based on frequencies of HLA molecules predicted to bind to peptides at the iedb.org website;
d. synthesizing the corresponding peptides;
confirming by testing for SARS-CoV-2-specific responses in sensitized individuals that have had mild COVID-19 disease (no hospitalization) and have recovered well.


SEQUENCE LISTING (82 sequences)

SEQ ID NO: 1 - protein, 15 aa, SARS-CoV-2
FGEVFNATRFASVYA

SEQ ID NO: 2 - protein, 20 aa, SARS-CoV-2
GNYNYLYRLFRKSNLKPFER

SEQ ID NO: 3 - protein, 20 aa, SARS-CoV-2
VGYQPYRVVVLSFELLHAPA

SEQ ID NO: 4 - protein, 20 aa, SARS-CoV-2
IPFAMQMAYRFNGIGVTQNV

SEQ ID NO: 5 - protein, 18 aa, SARS-CoV-2
QLIRAAEIRASANLAATK

SEQ ID NO: 6 - protein, 20 aa, SARS-CoV-2
KAHFPREGVFVSNGTHWFVT

SEQ ID NO: 7 - protein, 18 aa, SARS-CoV-2
SFYVYSRVKNLNSSRVPD

SEQ ID NO: 8 - protein, 20 aa, SARS-CoV-2
NRFLYIIKLIFLWLLWPVTL

SEQ ID NO: 9 - protein, 19 aa, SARS-CoV-2
ACFVLAAVYRINWITGGIA

SEQ ID NO: 10 - protein, 16 aa, SARS-CoV-2
ASFRLFARTRSMWSFN

SEQ ID NO: 11 - protein, 16 aa, SARS-CoV-2
DQIGYYRRATRRIRGG

SEQ ID NO: 12 - protein, 17 aa, SARS-CoV-2
ALALLLLDRLNQLESKM

SEQ ID NO: 13 - protein, 19 aa, SARS-CoV-2
SAFFGMSRIGMEVTPSGTW

SEQ ID NO: 14 - protein, 15 aa, SARS-CoV-2
TSLLVLVQSTQWSLF

SEQ ID NO: 15 - protein, 15 aa, SARS-CoV-2
SDFVRATATIPIQAS

SEQ ID NO: 16 - protein, 20 aa, SARS-CoV-2
INFVRIIMRLWLCWKCRSKN

SEQ ID NO: 17 - protein, 16 aa, SARS-CoV-2
AAIVFITLCFTLKRKT

SEQ ID NO: 18 - protein, 17 aa, SARS-CoV-2
MKFLVFLGIITTVAAFH

SEQ ID NO: 19 - protein, 15 aa, SARS-CoV-2
SKWYIRVGARKSAPL

SEQ ID NO: 20 - protein, 9 aa, SARS-CoV-2
GVYFASTEK

SEQ ID NO: 21 - protein, 9 aa, SARS-CoV-2
YLQPRTFLL

SEQ ID NO: 22 - protein, 9 aa, SARS-CoV-2
MIAQYTSAL

SEQ ID NO: 23 - protein, 9 aa, SARS-CoV-2
SIIAYTMSL

SEQ ID NO: 24 - protein, 9 aa, SARS-CoV-2
FIAGLIAIV

SEQ ID NO: 25 - protein, 9 aa, SARS-CoV-2
FAPSASAFF

SEQ ID NO: 26 - protein, 9 aa, SARS-CoV-2
LALLLLDRL

SEQ ID NO: 27 - protein, 9 aa, SARS-CoV-2
LLLDRLNQL

SEQ ID NO: 28 - protein, 9 aa, SARS-CoV-2
ATSRTLSYY

SEQ ID NO: 29 - protein, 10 aa, SARS-CoV-2
TLACFVLAAV

SEQ ID NO: 30 - protein, 9 aa, SARS-CoV-2
YYKKDNSYF

SEQ ID NO: 31 - protein, 9 aa, SARS-CoV-2
ASMPTTIAK

SEQ ID NO: 32 - protein, 9 aa, SARS-CoV-2
AELAKNVSL

SEQ ID NO: 33 - protein, 9 aa, SARS-CoV-2
TISLAGSYK

SEQ ID NO: 34 - protein, 9 aa, SARS-CoV-2
GEAANFCAL

SEQ ID NO: 35 - protein, 9 aa, SARS-CoV-2
FLNRFTTTL

SEQ ID NO: 36 - protein, 9 aa, SARS-CoV-2
VYMPASWVM

SEQ ID NO: 37 - protein, 9 aa, SARS-CoV-2
CTDDNALAY

SEQ ID NO: 38 - protein, 9 aa, SARS-CoV-2
AESHVDTDL

SEQ ID NO: 39 - protein, 9 aa, SARS-CoV-2
KSAGFPFNK

SEQ ID NO: 40 - protein, 9 aa, SARS-CoV-2
LTNDNTSRY

SEQ ID NO: 41 - protein, 9 aa, SARS-CoV-2
TYACWHHSI

SEQ ID NO: 42 - protein, 9 aa, SARS-CoV-2
GFDYVYNPF

SEQ ID NO: 43 - protein, 9 aa, SARS-CoV-2
KRVDWTIEY

SEQ ID NO: 44 - protein, 9 aa, SARS-CoV-2
HANEYRLYL

SEQ ID NO: 45 - protein, 9 aa, SARS-CoV-2
VVNARLRAK

SEQ ID NO: 46 - protein, 34 aa, SARS-CoV-2
KACFVLAAVYRINWITGGIAGPGPGKPRQKRTAT

SEQ ID NO: 47 - protein, 32 aa, SARS-CoV-2
KASFRLFARTRSMWSFNVGPGPGLALLLLDRL

SEQ ID NO: 48 - protein, 36 aa, SARS-CoV-2
KNRFLYIIKLIFLWLLWPVTLKGPGPGGVYFASTEK

SEQ ID NO: 49 - protein, 31 aa, SARS-CoV-2
KAAIVFITLCFTLKRKTGPGPGKSAGFPFNK

SEQ ID NO: 50 - protein, 35 aa, SARS-CoV-2
KIPFAMQMAYRFNGIGVTQNVGPGPGFLNRFTTTL

SEQ ID NO: 51 - protein, 32 aa, SARS-CoV-2
KQLIRAAEIRASANLAATGPGPGHANEYRLYL

SEQ ID NO: 52 - protein, 32 aa, SARS-CoV-2
KMKFLVFLGIITTVAAFHGPGPGYYKKDNSYF

SEQ ID NO: 53 - protein, 35 aa, SARS-CoV-2
KINFVRIIMRLWLCWKCRSKNGPGPGLTNDNTSRY

SEQ ID NO: 54 - protein, 85 aa, SARS-CoV-2
ALALLLLDRLNQLESKMGPGPGDQIGYYRRATRRIRGGGPGPGQLIRAAEIRASANLAATKGPGPGSAFFGMSRIGMEVTPSGTW

SEQ ID NO: 55 - protein, 83 aa, SARS-CoV-2
IPFAMQMAYRFNGIGVTQNVGPGPGTSLLVLVQSTQWSLFGPGPGSKWYIRVGARKSAPLGPGPGSFYVYSRVKNLNSSRVPD

SEQ ID NO: 56 - protein, 173 aa, SARS-CoV-2
ALALLLLDRLNQLESKMGPGPGDQIGYYRRATRRIRGGGPGPGQLIRAAEIRASANLAATKGPGPGSAFFGMSRIGMEVTPSGTWGPGPGIPFAMQMAYRFNGIGVTQNVGPGPGTSLLVLVQSTQWSLFGPGPGSKWYIRVGARKSAPLGPGPGSFYVYSRVKNLNSSRVPD

SEQ ID NO: 57 - protein, 149 aa, SARS-CoV-2
LLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYF

SEQ ID NO: 58 - protein, 392 aa, SARS-CoV-2
LLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYFGPGPGALALLLLDRLNQLESKMGPGPGDQIGYYRRATRRIRGGGPGPGQLIRAAEIRASANLAATKGPGPGIPFAMQMAYRFNGIGVTQNVGPGPGFGEVFNATRFASVYAGPGPGSDFVRATATIPIQASGPGPGTSLLVLVQSTQWSLFGPGPGKAHFPREGVFVSNGTHWFVTGPGPGSKWYIRVGARKSAPLGPGPGSAFFGMSRIGMEVTPSGTWGPGPGSFYVYSRVKNLNSSRVPD

SEQ ID NO: 59 - protein, 438 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 60 - protein, 438 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNK

SEQ ID NO: 61 - protein, 438 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 62 - protein, 657 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 63 - protein, 319 aa, SARS-CoV-2
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKEAAAKEAAAKEAAAKALALLLLDRLNQLESKMGPGPGDQIGYYRRATRRIRGGGPGPGQLIRAAEIRASANLAATKGPGPGSAFFGMSRIGMEVTPSGTW

SEQ ID NO: 64 - protein, 383 aa, SARS-CoV-2
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKEAAAKEAAAKEAAAKLLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYF

SEQ ID NO: 65 - protein, 626 aa, SARS-CoV-2
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKEAAAKEAAAKEAAAKLLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYFGPGPGALALLLLDRLNQLESKMGPGPGDQIGYYRRATRRIRGGGPGPGQLIRAAEIRASANLAATKGPGPGIPFAMQMAYRFNGIGVTQNVGPGPGFGEVFNATRFASVYAGPGPGSDFVRATATIPIQASGPGPGTSLLVLVQSTQWSLFGPGPGKAHFPREGVFVSNGTHWFVTGPGPGSKWYIRVGARKSAPLGPGPGSAFFGMSRIGMEVTPSGTWGPGPGSFYVYSRVKNLNSSRVPD

SEQ ID NO: 66 - protein, 538 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKEAAAKEAAAKEAAAKALALLLLDRLNQLESKMGPGPGDQIGYYRRATRRIRGGGPGPGQLIRAAEIRASANLAATKGPGPGSAFFGMSRIGMEVTPSGTW

SEQ ID NO: 67 - protein, 536 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKEAAAKEAAAKEAAAKIPFAMQMAYRFNGIGVTQNVGPGPGTSLLVLVQSTQWSLFGPGPGSKWYIRVGARKSAPLGPGPGSFYVYSRVKNLNSSRVPD

SEQ ID NO: 68 - protein, 626 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKEAAAKEAAAKEAAAKALALLLLDRLNQLESKMGPGPGDQIGYYRRATRRIRGGGPGPGQLIRAAEIRASANLAATKGPGPGSAFFGMSRIGMEVTPSGTWGPGPGIPFAMQMAYRFNGIGVTQNVGPGPGTSLLVLVQSTQWSLFGPGPGSKWYIRVGARKSAPLGPGPGSFYVYSRVKNLNSSRVPD

SEQ ID NO: 69 - protein, 626 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKEAAAKEAAAKEAAAKALALLLLDRLNQLESKMGPGPGDQIGYYRRATRRIRGGGPGPGQLIRAAEIRASANLAATKGPGPGSAFFGMSRIGMEVTPSGTWGPGPGIPFAMQMAYRFNGIGVTQNVGPGPGTSLLVLVQSTQWSLFGPGPGSKWYIRVGARKSAPLGPGPGSFYVYSRVKNLNSSRVPD

SEQ ID NO: 70 - protein, 845 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKEAAAKEAAAKEAAAKLLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYFGPGPGALALLLLDRLNQLESKMGPGPGDQIGYYRRATRRIRGGGPGPGQLIRAAEIRASANLAATKGPGPGIPFAMQMAYRFNGIGVTQNVGPGPGFGEVFNATRFASVYAGPGPGSDFVRATATIPIQASGPGPGTSLLVLVQSTQWSLFGPGPGKAHFPREGVFVSNGTHWFVTGPGPGSKWYIRVGARKSAPLGPGPGSAFFGMSRIGMEVTPSGTWGPGPGSFYVYSRVKNLNSSRVPD

SEQ ID NO: 71 - protein, 845 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKEAAAKEAAAKEAAAKLLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYFGPGPGALALLLLDRLNQLESKMGPGPGDQIGYYRRATRRIRGGGPGPGQLIRAAEIRASANLAATKGPGPGIPFAMQMAYRFNGIGVTQNVGPGPGFGEVFNATRFASVYAGPGPGSDFVRATATIPIQASGPGPGTSLLVLVQSTQWSLFGPGPGKAHFPREGVFVSNGTHWFVTGPGPGSKWYIRVGARKSAPLGPGPGSAFFGMSRIGMEVTPSGTWGPGPGSFYVYSRVKNLNSSRVPD

SEQ ID NO: 72 - protein, 845 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKEAAAKEAAAKEAAAKLLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYFGPGPGALALLLLDRLNQLESKMGPGPGDQIGYYRRATRRIRGGGPGPGQLIRAAEIRASANLAATKGPGPGIPFAMQMAYRFNGIGVTQNVGPGPGFGEVFNATRFASVYAGPGPGSDFVRATATIPIQASGPGPGTSLLVLVQSTQWSLFGPGPGKAHFPREGVFVSNGTHWFVTGPGPGSKWYIRVGARKSAPLGPGPGSAFFGMSRIGMEVTPSGTWGPGPGSFYVYSRVKNLNSSRVPD

SEQ ID NO: 73 - protein, 402 aa, SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKPKPKPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPK

SEQ ID NO: 74 - protein, 402 aa, SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKPKPKPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPK

SEQ ID NO: 75 - protein, 535 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKKPKPKPALALLLLDRLNQLESKMGPGPGDQIGYYRRATRRIRGGGPGPGQLIRAAEIRASANLAATKGPGPGSAFFGMSRIGMEVTPSGTWKPKPKPRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 76 - protein, 533 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKGGGGSALALLLLDRLNQLESKMGPGPGDQIGYYRRATRRIRGGGPGPGQLIRAAEIRASANLAATKGPGPGSAFFGMSRIGMEVTPSGTWGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 77 - protein, 533 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKKPKPKPIPFAMQMAYRFNGIGVTQNVGPGPGTSLLVLVQSTQWSLFGPGPGSKWYIRVGARKSAPLGPGPGSFYVYSRVKNLNSSRVPDKPKPKPRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNK

SEQ ID NO: 78 - protein, 531 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGGGGSIPFAMQMAYRFNGIGVTQNVGPGPGTSLLVLVQSTQWSLFGPGPGSKWYIRVGARKSAPLGPGPGSFYVYSRVKNLNSSRVPDGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNK

SEQ ID NO: 79 - protein, 599 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKKPKPKPLLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYFKPKPKPRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 80 - protein, 842 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKKPKPKPLLLDRLNQLGPGPGGVYFASTEKGPGPGTISLAGSYKGPGPGKRVDWTIEYGPGPGKPRQKRTATGPGPGLTNDNTSRYGPGPGAELAKNVSLGPGPGGFDYVYNPFGPGPGFLNRFTTTLGPGPGKSAGFPFNKGPGPGYYKKDNSYFGPGPGALALLLLDRLNQLESKMGPGPGDQIGYYRRATRRIRGGGPGPGQLIRAAEIRASANLAATKGPGPGIPFAMQMAYRFNGIGVTQNVGPGPGFGEVFNATRFASVYAGPGPGSDFVRATATIPIQASGPGPGTSLLVLVQSTQWSLFGPGPGKAHFPREGVFVSNGTHWFVTGPGPGSKWYIRVGARKSAPLGPGPGSAFFGMSRIGMEVTPSGTWGPGPGSFYVYSRVKNLNSSRVPDKPKPKPRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNK

SEQ ID NO: 81 - protein, 491 aa, SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKPKPKALALLLLDRLNQLESKMGPGPGDQIGYYRRATRRIRGGGPGPGQLIRAAEIRASANLAATKGPGPGSAFFGMSRIGMEVTPSGTWPKPKPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPK

SEQ ID NO: 82 - protein, 489 aa, SARS-CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKPKPKIPFAMQMAYRFNGIGVTQNVGPGPGTSLLVLVQSTQWSLFGPGPGSKWYIRVGARKSAPLGPGPGSFYVYSRVKNLNSSRVPDPKPKPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPK